(19) United States
(12) Patent Application Publication
(10) Pub. No.: US 2014/0256843 A1
(43) Pub. Date: Sep. 11, 2014

(54) BIOMATERIAL FOR OSTEOSYNTHESIS

(71) Applicants: TEKNIMED, L'Union (FR); ARKEMA FRANCE, Colombes (FR); UNIVERSITE PAUL SABATIER, Toulouse (FR)

(72) Inventors: Cyril SENDER, TOULOUSE (FR); Colette LACABANNE, TOULOUSE (FR); Alain BERNES, FONSEGRIVES (FR); Michel GLOTIN, SAINT CLOUD (FR)

(73) Assignees: TEKNIMED, L'Union (FR); ARKEMA FRANCE, Colombes (FR); UNIVERSITE PAUL SABATIER, Toulouse (FR)

(21) Appl. No.: 14/242,354

(22) Filed: Apr. 1, 2014

Related U.S. Application Data
(63) Continuation of application No. 11/970,596, filed on Jan. 8, 2008.

(30) Foreign Application Priority Data
Jan. 7, 2008 (FR) 08 00077

(51) Int. Cl. A61L 27/46

(52) U.S. Cl. CPC A61L 27/46
USPC 523/115

(57) ABSTRACT

A biomaterial for the manufacture of osteosynthesis articles is provided. The biomaterial contains a semi-aromatic polyamide matrix along with a reinforcing means selected to reinforce the matrix. The biomaterial has dynamic mechanical properties analogous to calcified tissue.

### FIELD OF THE INVENTION

The present invention relates to a biomaterial for the manufacture of osteosynthesis articles provided with dynamic mechanical properties analogous to those of bone.

### BACKGROUND OF THE INVENTION

Numerous bone complications, of pathological or traumatological origin, are indications for the use of prosthetic biomaterials. Orthopaedic surgery represents a growing market because of the ageing of the population, pathologies such as bone tumours and osteoporosis, and obesity which affects more and more people throughout the world.

Bone material is a hybrid composite composed of an organic phase, a mineral phase and water, representing on average 22, 69 and 9% by weight respectively in adult mammals [Lee 1981, Banks 1993]. The organic phase is made up of 90% fibrillar substance (predominantly collagen) and 10% other minority organic compounds which form the so-called fundamental or interfibrillar substance [Fisher 1985, Toppets 2004]. On a molecular scale, collagen, the major component of the organic bone phase, is a protein with which are associated various structuration levels. Generally, collagen is made up of polypeptidic chains of 1052 to 1060 residues linked by peptidic connections (CO—NH). This organic phase is at the origin of the viscoelasticity of the calcified tissue. The mineral phase is composed of calcium phosphate crystals with a chemical composition close to hydroxyapatite Ca₁₀(PO₄)₆(OH)₂ [Rey 1990]. It is these crystals which give the calcified tissues their elasticity and their rigidity.

Two principal types of bone tissues exist: cortical or compact tissue, and trabecular or spongy tissue, representing 80 and 20% respectively of the skeletal weight [Bronner 1999]. Compact bone, also called Haversian bone, appears as a solid, dense mass; it is principally responsible for the function of mechanical support. The basic unit of the cortical bone is an assembly of 20 to 30 concentric strips forming what is called an osteon system with an average diameter of 200 to 250 μm in man [Cowin 2001]. These osteons are aligned in parallel in the axis of the bone (along the lines of the field of mechanical stress) and are linked by means of older strip-like interstitial bone arising from the reabsorption of old osteons.

Bone is a living material and undergoes multiple morphological changes during its growth, its constant renewal (remodelling), its ageing, and finally in the course of pathological disorders (osteoporosis, osteosarcoma . . . ) or traumatological disorders (fissures, fractures). The various phases in the formation and reabsorption of bone tissues involve hormones and all of the cellular material. The balance between the dynamic processes of bone remodelling is governed by the fields of stresses and the deformations undergone by the skeleton [Wolff 1892]. The disturbance or the permanent modification of the mechanical environment of a bone region terminates in a redistribution of the physiological field of stress. The response of the organism is to change the geometry of the bone in order to adapt it to its new mechanical environment. This situation is encountered when an osteosynthesis device is used in orthopaedic surgery [O'Doherty 1995].

The mechanical properties of bone tissues have been the subject-matter of a large number of publications. Bone tissue was initially considered to be a resilient material characterised by its behaviour in a static regime. In physiological conditions, it is subjected to dynamic stresses (physiological frequencies between 0.1 and 10 Hz): it then has a viscoelastic behaviour. Dynamic Mechanical Spectrometry (DMS) permits this to be defined: a sinusoidal deformation γ (represented by γ*) is applied and causes the establishment of a sinusoidal stress σ (represented by σ*) in the sample, with a dephasing denoted by δ. The complex mechanical shearing modulus G* is thus determined:

G*=σ*/γ*

It can also be defined as being the sum of the elastic or preservative modulus G′ and of the dissipative or losses modulus G″:

G*=G′+G″

The ratio of G″ to G′, denoted by tan δ, is the mechanical energy loss factor.

For the measured values to be representative of the physiological state, it is necessary for the measures to be effected in physiological liquid. The values of the mechanical modulus of shearing G′, referenced in the literature, go from 100 MPa to 10 GPa; the values reported for tan δ are in the order of 10⁻².

With the aim of getting as close as possible to the mechanical behaviour of bone tissue, it is necessary to define a biomaterial, i.e. a non-living material used in a medical device intended to interact with biological systems [Chester Conference of the European Society of Biomaterials, 1986]. In the large family of biomaterials used in the field of orthopaedic surgery, metal alloys, ceramics and materials based on polymer are well represented. Each of these three groups has advantages and disadvantages which are their own.

At the present time, the great majority of materials used for this type of application (total hip prostheses, total knee prostheses, osteosynthesis articles . . . ) are metallic materials with a high modulus, comparative to that of the cortical bone onto which they are secured. The field of physiological stress is deflected by this device which then supports all of the stresses and thus achieves a shielding of the stresses, called “stress shielding” in the literature [Brown 1981]. This is the phenomenon which induces a negative remodelling balance, hence a reabsorption: the areas of the bone which are no longer fulfilling their mechanical support role are reabsorbed by the organism, and the risks of fracture after withdrawal of the implant or of its detachment in the medium term are increased [Vaughan 1970, Uthoff 1971, Tonino 1976, Paavolainen 1978, Slätis 1978, Bradley 1980, Cook 1982, Uthoff 1983, Claes 1989, Huiskes 1989, Damien 1991, Huiskes 1995].

It has been shown that the use of biomaterials having resilient properties closer to the cortical bone permits the processes of osteogenesis to be accelerated [Robbins 2004]. The reduction in the mechanical protection of the bone by the use of semi-rigid implants permits the bone matter to be induced more while sufficiently reducing mobility at the level of the fracture sites or of the interfaces between the osteoconductive ceramic material and the vertebral discs in the case of vertebral arthrodeses. This has been shown recently by comparing the fusion rate of vertebrae with the aid of intersomatic cages formed from titanium and from biodegradable polymer [Pflugmacher 2004]. With the latter, the fusion of the vertebrae is more rapid: the mechanical stresses acting at the level of the vertebral discs are not totally deflected by the cages and are transmitted to the osteoconductive material placed in their centre. An intimate and dynamic contact then exists between this material and the vertebrae, accelerating osteogenesis and fusion.

Bioceramic materials such as zirconia, alumina, calcium phosphates or indeed metal prostheses based on titanium or other alloys, have moduli of elasticity widely superior to that of cortical or spongy bone. For example, titanium or the titanium alloy called Ti-6A14V, used for the manufacture of a total hip prosthesis, has a Young modulus in the order of 100 GPa, and the stainless steel AISI 316LTi has a Young modulus of 140 GPa [Long 1998]. Bioceramic materials also have high moduli of elasticity (several hundreds of GPa) and are fragile [Ramakrishna 2001]. It is to be noted that it is the rigidity of the implants which is responsible for the level of deflection of the mechanical stresses [Brown 1979, Claes 1989]. This result has been at the origin of the development of metal implants which are less thick or porous to reduce their rigidity. But then, the properties of resistance to fatigue reduce, and the implanted devices become unviable.

Compact bone has a loss factor in the order of 10⁻². This characteristic is physiologically fundamental, since it is this which quantifies the capability of the bone to absorb a portion of the mechanical energy generated during our daily activities and necessary for its remodelling. Rigid biomaterials have a mechanical loss factor tan δ less than 10⁻³, i.e. 3.6.10⁻⁶ for certain aluminium alloys [Garner 2000].

Although the current metal biomaterials, called “low modulus”, are close to the mechanical properties of bone, they still remain much more structural. The only medical devices which would permit the mechanical protection of the bone tissues to be avoided are semi-rigid materials. This is the concept of so-called “analogous” biomaterials, introduced by Bonfield in the eighties [Bonfield 1981]. An incontrovertible family in this field is that of the polymer materials, well-known for their capability to absorb by viscous dissipation. In order to be mechanically biocompatible, the macromolecular systems must possess both high resilient properties and mechanical absorption properties comparable with bone matter.

Osteosynthesis devices based on non-bioreabsorbable synthetic polymers have been the subject-matter of tests on animals. Since these devices generally have intrinsic mechanical properties inferior to those of bone, they were reinforced. The composites obtained have a viscous behaviour similar to calcified tissues, and moduli of elasticity generally lower than bone. By way of illustration, discs formed from polytrifluoromonochloroethylene (PTFCE) of Tonino et al. [Tonino 1976], and semi-rigid osteosynthesis plates based on polysulphone/graphite and epoxy/glass of Bradley et al. [Bradley 1977] are to be mentioned. Plates formed from polymers charged with carbon fibres, having moduli of elasticity going from 2 to 3.5 GPa, have been tested for static torsion [Claes 1980]. The implantation of these discs on animals has posed problems of resistance to rupture, and has not been conclusive.

The first cases of implantation of semi-rigid osteosynthesis discs on human beings were reported by Tayton et al. [Tayton 1982]. Multiaxial discs formed from epoxy resin reinforced with carbon fibres were implanted on patients suffering from fractures: the bone repairs itself rapidly and reaches normal rigidity in only 25 weeks. For securing a fractured tibia, Tayton and Bradley will go so far as to propose an optimum rigidity of the osteosynthesis plate of 2.0 N.m by degrees [Tayton 1983].

Numerous other composites have been developed and studied for applications in orthopaedics. Among these are found polymer matrices charged with particles of HAp, as in the case of High Density Polyethylene [Bonfield 1981, Tanner 1992, Wang 1994, Deb 1996, Wang 1998, Roeder 2003], polylactides [Verheyen 1992, Kikuchi 1997, Zhang 1999, Shikinami 1999, Ignjatovic 1599, Durucan 2000], PMMA [Ravaglioli 1992, Kazuhiko 1992, Harper 2000], acrylic Polyacid [Liou 2003] . . . Others have been reinforced by means of long or short carbon fibres. Although this element has excellent biocompatibility (totally inert), the release in vivo of worn particles into the surrounding tissues has given bad results [Claes 1983]. The ends of carbon fibres at the surface of the implants are extremely abrasive and irritating [Evans 1998]. Wan et al. have also shown that, despite the chemical inertia of silicon carbide fibres, their level of cytotoxicity in direct contact with cells is high [Wan 1993].

The use of structural materials capable of absorbing a portion of the mechanical energy is therefore no longer to be demonstrated. The concept of semi-rigidity has long excited interest in the field of orthopaedics. But still today, metals and in particular implants based on titanium are widely used for lack of semi-rigid materials which have moduli of elasticity within the range of bone tissues.

In order to resolve the technical problem of resistance to the stress of the polymers used as biomaterials, the introduction of aromatic cycles in the chain structure of the polymer has been envisaged in order to increase its physical properties. Such materials, currently in development for automobile applications, have never been envisaged in the medical field, in view of the gulf between the problems encountered in these two fields.

Technical industrial polymers have been developed from aromatic polyamides since the sixties. One of the best-known is Kevlar or Poly-para-phenylene terephthalamide produced by the Du Pont company of Nemours in 1965. This material combines very high mechanical properties, associated with great capabilities of absorbing shocks, and excellent resistance to fatigue and to numerous solvents. Its applications are varied: aeronautical and aerospatial protective equipment (helmets, jackets), sports equipment . . . Since its mechanical properties are very high and its implementation is not simple, some industrialists have developed polyamides having an intermediate composition between that of aromatic polyamides and aliphatic polyamides, such as Polyamide 6 (PA6) or Polyamide 11 (PA11). These are so-called semi-aromatic polyamides SAPAs. Monitoring the relative content of aromatic cycle in the chain structure permits the physical properties of these polymers to be adjusted. Combining the remarkable shock-absorbing properties of the polyamides with the high mechanical and thermal properties of the aromatic polymers, the family of SAPAs permits a large number of applications to be satisfied. Active industrial research has led to the marketing of numerous SAPAs such as Cristamid® from Arkema based on PA12, IXEF® from Solvay, PA6/6T or Ultramid T® from BASF, Zytel® from Du Pont, PAST or Genestar® from Kuraray, Grilamid® from E.M.S, Trogamid® from Evonik . . .

In the biomedical field, only some aliphatic polyamides have been used in various applications such as suturing threads, dialysis membranes [Yamashita 1996], artificial skin [Bugmann 1998, Mei 2003], a cell culture medium [Catapano 1996], catheters, syringes . . . The biocompatibility of the polyamide materials is explained by the similarity of their chemical composition to natural proteins such as collagens [Risbud 2001, Jie 2001]. In fact, the amide groups contained in the polyamides are identical to the peptidic bonds in the proteins. The expression “natural polyamide” has even been used by Das et al. to qualify gelatine, a product arising from the denaturation of collagen [Das 2003].

The cytotoxicity level of the polyamide 6 used for the manufacture of cell culture supports in tissue engineering is low [Das 2003]. The implantation of polyamide 66, charged with hydroxyapatite, has given specifically interesting results in terms of biocompatibility [Xiang 2002]. However, its absorbency causes a drop in the mechanical properties in the hydrated state.

### SUMMARY OF THE INVENTION

In order to overcome the disadvantages of prior art, the present invention proposes a biomaterial for the manufacture of osteosynthesis articles having dynamic mechanical properties analogous to calcified tissue, characterised in that it includes a hydrophobic semi-aromatic polyamide matrix and at least one reinforcing means.

The term reinforcing means denotes any compound capable of optimising the mechanical properties of the matrix. Of variable morphology, the reinforcing means used in the present invention may have a particular appearance, that is to say with dimensions in the same order of size, i.e. between 10 nm and 100 μm.

The size of the reinforcing particles is a crucial factor for obtaining the reinforcing effect: the higher the developed surface between the matrix and the reinforcing means, the better will be the transfer of mechanical stress. Thus, the use of particles of nanometric dimensions permits the contact surface between the two phases to be increased considerably. One particularly advantageous shape for the particular reinforcing means consists of needles or strips which can be combined.

In the case of a non-particular reinforcing means, a fibrous appearance is also covered by the present invention. The reinforcing means is then defined by its shape factor Length (L) relative to diameter (d) with values greater than 10. The use of reinforcing means having a high shape factor optimises the mechanical properties of the composites.

In a preferred manner, the reinforcing means will consist of inorganic compounds selected from glasses, silicates, calcium phosphates and a mixture thereof.

In a biomimetic sense, the material selected to reinforce the polyamide matrix is hydroxyapatite or HAp. The hydrophilic (polar) character of the apatitic materials permits the formation of physical bonds with the polar groups of the polyamide matrix, which bonds are indispensable for the transfer of the mechanical loads from the matrix to the reinforcing means.

The reinforcing means may also be an organic compound, selected preferably from polyamides or carbon and a mixture thereof.

### BRIEF DESCRIPTION OF THE DRAWINGS

FIG. 1 illustrates the preservative modulus G′ as a function of the frequency of a biomaterial according to the invention, comprising a semi-aromatic polyamide matrix based on PA11/10,T and a reinforcing rate of 20% of Hap.

FIG. 2 illustrates the mechanical energy loss factor tan δ as a function of the frequency of a biomaterial according to the invention, comprising a semi-aromatic polyamide matrix based on PA11/10,T and a reinforcing means rate of 20% of Hap.

FIG. 3 illustrates the results of MTT and neutral Red tests on extracts of PA11/10,T at 100%, 50%, 10% and 1%.

FIG. 4 illustrates the rectangular torsion device in the liquid cell fitted with a thermostat used for measuring the dynamic mechanical modulus of shearing G*.

### DETAILED DESCRIPTION OF THE INVENTION

The semi-aromatic polyamide matrix according to the invention includes at least one homopolyamide of the formula Y.Ar with:
Y representing an element comprising at least one saturated, linear or branched aliphatic and/or cycloaliphatic diamine having preferably between 4 and 20 carbon atoms, and
Ar representing an element comprising at least one possibly substituted aromatic carboxylic diacid having preferably between 8 and 22 carbon atoms, said aromatic carboxylic diacid advantageously being a phthalic acid, the phthalic acid preferably being selected from terephthalic acid, isophthalic acid, orthophthalic acid and their mixtures.

It may also include at least one copolyamide of the formula X/Y.Ar with:
Y representing an element comprising at least one saturated, linear or branched aliphatic and/or cycloaliphatic diamine having preferably between 4 and 20 carbon atoms,
Ar representing an element comprising at least one possibly substituted aromatic carboxylic diacid having preferably between 8 and 22 carbon atoms, said aromatic carboxylic diacid advantageously being a phthalic acid, the phthalic acid preferably being selected from terephthalic acid, isophthalic acid, orthophthalic acid and their mixtures, and
X representing:
   either an element comprising at least one lactam and/or at least one carboxylic alpha-omega aminoacid, the lactam and/or the carboxylic alpha-omega aminoacid having preferably between 6 and 18 carbon atoms,
   or an element U.V arising from the condensation of at least one diamine U with at least one carboxylic diacid V,
      the linear or branched diamine U being selected from an aliphatic diamine, a cycloaliphatic diamine, an aromatic diamine and their mixtures and having preferably between 4 and 20 carbon atoms, and
      the linear or branched carboxylic diacid V being selected from an aliphatic diacid, a cycloaliphatic diacid, an aromatic diacid and their mixtures, and having preferably between 6 and 20 carbon atoms.

Preferably, the number of carbon atoms of one at least of the elements X and Y is between 6 and 12.

Y and U are preferably selected from the following group: 1,6-hexamethylene diamine 1,9-nonane diamine, 2-methyl-1,8-octane diamine, 1,10-decane diamine, 1,12-dodecane diamine, and their mixtures.

X preferably comprises lactam 12, amino-11-undecanoic acid, amino-12-dodecanoic acid and their mixtures.

V is preferably selected from the following group: adipic acid, suberic acid, azelaic acid, sebacic acid, 1,12-dodecanedioic diacid, brassylic acid, 1,14-tetradecanedioic diacid, terephthalic acid, isophthalic acid, naphthalene dicarboxylic acid, and their mixtures.

The molar proportions of X relative to Y (or Ar) are, for Y=1, 0≦X≦0.7, preferably 0≦X≦0.5.

The diamines Y and U may be identical or not.

In the formulae Y.Ar and X/Y.Ar, the expressions “at least one diamine” and “at least one diacid” denote, respectively and independently of each other, “one, two or three diamine(s)” and “one, two or three diacid(s)”.

The biomaterial according to the present invention includes up to 70% by weight of reinforcing means relative to the total weight of the biomaterial. Although optional, it may include a surfactant agent or a mixture of surfactant agents, an amphiphilic molecule or a mixture of amphiphilic molecules or any other compatibilising agent or mixture of compatibilising agents. By way of example, “glycol” polyethylenes, fatty acids such as palmitic acid, . . . can be mentioned.

In order to optimise the mechanical properties of the biomaterial, said material must include a percentage of added water of less than 5% by total weight. If necessary, a complementary step of drying the biomaterial is carried out in order to attain this percentage of water.

The biomaterial thus defined is characterised by dynamic mechanical properties analogous to calcified tissue. These properties correspond to a significant level of viscoelasticity at physiological temperatures (37° C.) and frequencies (0.1 to 10 Hz) defined by a preservative modulus and a mechanical energy loss factor in the order of those of the calcified tissue.

The values of the preservative modulus, represented by G′, corresponding to the biomaterial according to the invention, are thus between 100 MPa and 10 GPa, in the shearing mode.

The values of the mechanical energy loss factor, represented by tan δ, are greater than 10⁻³ in the shearing mode.

The biomaterial according to the present invention is particularly intended for the manufacture of osteosynthesis devices or dental prostheses. More widely, it can be used in any medical application which requires compounds provided with mechanical properties close to bone tissue.

The properties of the biomaterial according to the invention are shown by the following Figures:

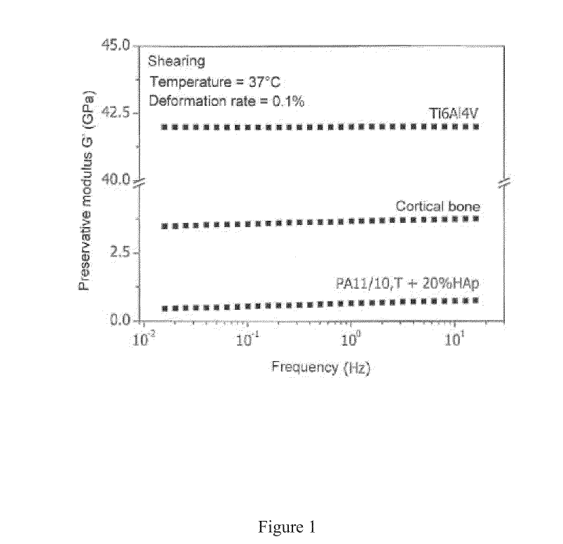

FIG. 1: illustrates the preservative modulus G′ as a function of the frequency of a biomaterial according to the invention, comprising a semi-aromatic polyamide matrix based on PA11/10,T and a reinforcing rate of 20% of HAp. These values are compared with those of a cortical bone as well as a material formed from the Ti6A14V alloy.

It is to be noted that the preservative modulus G′ of the biomaterial according to the invention is in the value zone of that of the cortical bone, while that of the material formed from the Ti6A14V alloy is ten times higher.

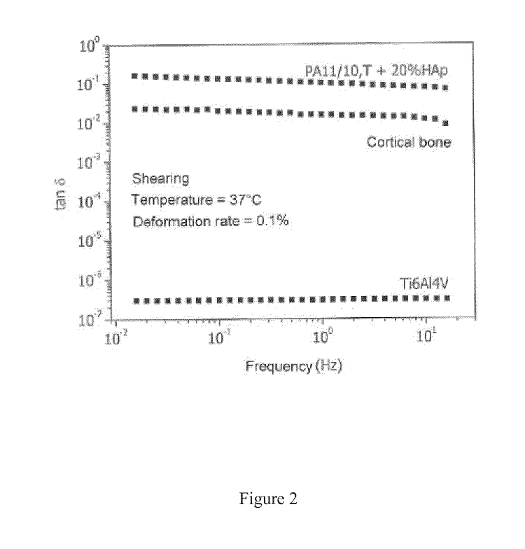

FIG. 2: illustrates the mechanical energy loss factor tan δ as a function of the frequency of a biomaterial according to the invention, comprising a semi-aromatic polyamide matrix based on PA11/10,T and a reinforcing means rate of 20% of HAp. These values are compared with those of a cortical bone as well as a material formed from the Ti6A14V alloy.

The mechanical energy loss factor of the biomaterial according to the invention is in the value zone of that of the cortical bone, while that of the material formed from the Ti6A14V alloy is very far removed therefrom.

The Examples which follow are intended to illustrate the present invention without limiting the scope thereof.

### EXAMPLE 1

Implementation of a Biomaterial According to the Invention in Dispersion by the Solvent Method
Solvent substitution:
Since the polyamides are not able to be dissolved in the DMAc when the medium contains water, the water of the nHAp/water suspension is substituted by DMAc.
Disagglomeration of the particles of nHAp:
The desired quantity of nHAp/DMAc suspension is agitated and sonified with ultrasound by means of a 500 W/20 kHz ultrasound probe from Sonics with a vibration amplitude fixed at 95% of the maximum amplitude of the apparatus. The birth then the explosion of microbubbles within the suspension causes the release of considerable energy (cavitation phenomenon), ensuring intense agitation of the medium which permits the agglomerates to be crushed.
Dissolving of the SAPA:
The desired quantity of SAPA is poured into the nHAp suspension and dissolved.
Precipitation, filtration and washing of the nanocomposite:
Distilled water, a non-solvent of the polyamide, is added in order to precipitate the nanocomposite. A suspension of millimetric particles of nanocomposite in a liquid medium, a mixture of water and DMAc, is obtained. The affinity between the water and the DMAc is greater than that between the nanocomposite and the DMAc, so that the water, in large excess in the medium, is substituted for the DMAc in the nanocomposite [Kasowski 1994]. The entirety is then filtered on a Büchner filter and thoroughly washed in distilled water. The product obtained is a white paste heavily saturated with water. It is dried in a drying cupboard.
Grinding:
The dried nanocomposite is in the form of coarse centimetric aggregates. The injection of this matter requires a preliminary grinding stage. The nanocomposites are soaked in nitrogen, and a ZM100-type grinder from Retsch is used in order to obtain a fine powder.

### EXAMPLE 2

Study of the Cytotoxicity of a Semi-Aromatic Polyamide Used as a Biomaterial According to the Invention: PA11/10,T

The PA11/10,T, provided by the Arkema company, is in the form of slightly opaque granules. It is a statistic polymer synthesised by the polycondensation of three monomers, 11-aminoundecaneoic acid, decamethylene diamine and terephthalic acid. PA11/10,T is a semi-crystalline polymer having a glass transition temperature in the order of 80° C. and a fusion over a range of temperatures of 200/270° C., in dependence on the molar proportion of 11-aminoundecaneoic acid relative to that of decamethylene diamine (or terephthalic acid). PA11/10,T absorbs about 1.2 and 2% by weight of water when it is respectively kept in ambient conditions or hydrated to saturation in distilled water.

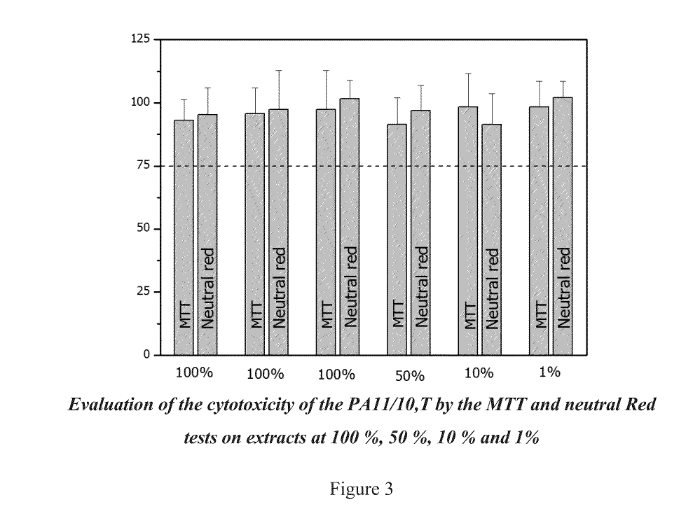

The cytotoxicity of PA11/10,T has been determined on human osteoprogenitor cell cultures produced from medullary stroma at the Laboratory of Biophysics of the Victor Segalen University in Bordeaux. A study of microbial precontamination before sterilisation, as well as the determination of the residual content of ethylene oxide after sterilisation, have shown that the PA11/10,T has been correctly conditioned and sterilised. The MTT test, characterising the metabolic activity of the cells, and the neutral Red test, which is evidence of the cell viability, were carried out. Extracts of the PA11/10,T at 100%, then diluted to 50, 10 and 1%, were tested. A material is considered to be cytotoxic if the values obtained are below 75% relative to the control cultures. The results of the tests, illustrated in FIG. 3, show that the PA11/10,T is not cytotoxic.

### EXAMPLE 3

Experimental Device Used for Measuring the Dynamic Mechanical Modulus of Shearing G*: Dynamic Mechanical Spectrometry (DMS)

The tests are carried out by means of an ARES rheometer from Thermal Analysis Instruments. The stress mode selected is rectangular torsion at an imposed deformation rate. A motor, integral with the lower end of the sample, applies a torsion movement, while the couple induced on the upper bit through the intermediary of the sample is recorded by a measuring cell. This torsion couple is then converted into stress.

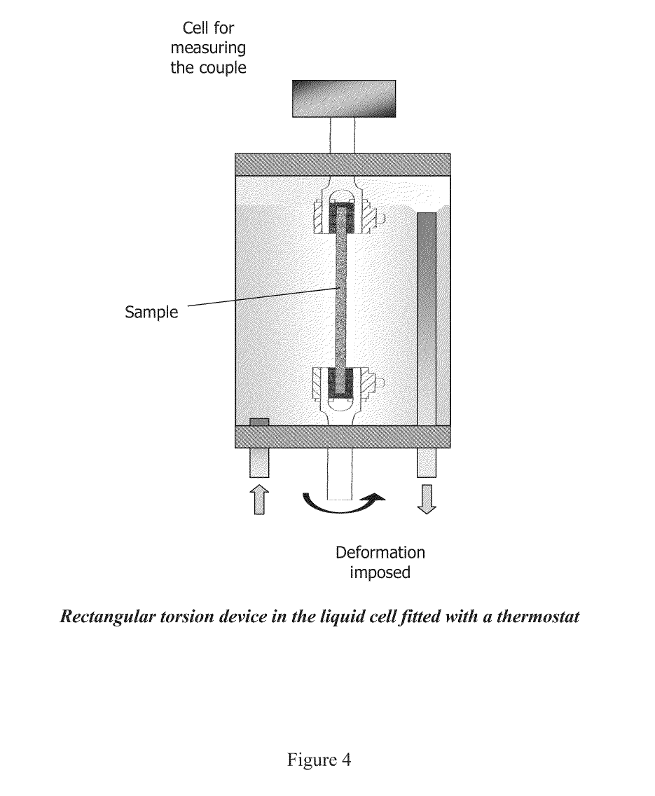

The samples may be stressed in air (in an oven) or immersed in an aqueous solution by means of a cell in which the fluid circulates (FIG. 4). In air, the temperature may vary between −140 and 300° C. The low temperatures are accessible by the use of a tank of liquid nitrogen. In an aqueous solution, the temperature range is restricted to 10/80° C. It is a Julabo F25 cryothermostat which then monitors the temperature of the circulating fluid.

The samples have a parallelepiped shape of width b, of thickness a and of length L, such that a<<b and b<L. A shape factor K is defined:

$$K = \frac{3 L}{{ab}^{3}} \times \frac{1}{1 - 0.63 \frac{b}{a}}$$

This factor permits the complex force σ*(ω) and the dynamic mechanical modulus G*(ω) to be connected:

$$G * ( ω ) = K σ ( ω ) = K \frac{{T}_{0}}{θ * ( ω )} {e}^{iδ ( ω )}$$

with T₀ being the torsion couple measured by the upper bit, and θ*(ω) being the angle of deformation of the lower end of the sample.

1-23. (canceled)

24. An osteosynthesis article comprising a biomaterial having dynamic mechanical properties analogous to calcified tissue, said biomaterial comprising:
at least one reinforcing means selected to reinforce a polyamide matrix by optimizing mechanical properties of said polyamide matrix, wherein,
the at least one reinforcing means is a calcium phosphate compound, and
the polyamide matrix is a semi-aromatic polyamide matrix.

25. The osteosynthesis article according to claim 24, wherein said reinforcing means possesses a particular appearance with a dimension of between 10 nm and 100 μm.

26. The osteosynthesis article according to claim 25, wherein said particular reinforcing means is in the form of needles and/or strips.

27. The osteosynthesis article according to claim 24, wherein said reinforcing means possesses a fibrous appearance with an L/d shape factor greater than 10.

28. The osteosynthesis article according to claim 24, wherein,
the semi-aromatic polyamide matrix includes at least one homopolyamide of the formula Y.Ar,
Y represents an element comprising at least one saturated, linear or branched aliphatic and/or cycloaliphatic diamine, and
Ar represents an element comprising at least one possibly substituted aromatic carboxylic diacid.

29. The osteosynthesis article according to claim 24, wherein
the semi-aromatic polyamide matrix includes at least one copolyamide of the formula X/Y.Ar,
Y represents an element comprising at least one saturated, linear or branched aliphatic and/or cycloaliphatic diamine,
Ar represents an element comprising at least one possibly substituted aromatic carboxylic diacid, and
X represents one of:
(i) an element comprising at least one lactam and/or at least one carboxylic alpha-omega aminoacid, the lactam and/or the carboxylic alpha-omega aminoacid, and
(ii) an element U.V arising from the condensation of at least one linear or branched diamine U with at least one linear or branched carboxylic diacid V,
the linear or branched diamine U is selected from the group consisting of an aliphatic diamine, a cycloaliphatic diamine, an aromatic diamine and their mixtures, and the linear or branched carboxylic diacid V is selected from the group consisting of an aliphatic diacid, a cycloaliphatic diacid, an aromatic diacid and their mixtures.

30. The osteosynthesis article according to claim 28, wherein the aromatic carboxylic diacid Ar is a phthalic acid selected from the group consisting of terephthalic acid, isophthalic acid, orthophthalic acid and their mixtures.

31. The osteosynthesis article according to claim 29, wherein the number of carbon atoms of one at least of the elements X and Y is between 6 and 12 carbon atoms.

32. The osteosynthesis article according to claim 29, wherein one at least of the element Y and of the diamine U is selected from the following group consisting of: 1,6-hexamethylene diamine, 1,9-nonane diamine, 2-methyl-1,8-octane diamine, 1,10-decane diamine, 1,12-dodecane diamine and their mixtures.

33. The osteosynthesis article according to claim 29, wherein X is selected from lactam 12 amino-11-undecanoic acid, amino-12-dodecanoic acid and their mixtures.

34. The osteosynthesis article according to claim 29, wherein V is selected from the group consisting of: adipic acid, suberic acid, azelaic acid, sebacic acid, 1,12-dodecanedioic diacid, brassylic acid, 1,14-tetradecanedioic diacid, terephthalic acid, isophthalic acid, naphthalene dicarboxylic acid and their mixtures.

35. The osteosynthesis article according to claim 29, wherein the molar proportions of X relative to Y (or Ar) are:
for Y=1,
0<X<0.7.

36. The osteosynthesis article according to claim 35, wherein the molar proportions of X relative to Y (or Ar) are:
for Y=1,
0<X<0.5.

37. The osteosynthesis article according to claim 29, wherein the diamines Y and U are identical.

38. The osteosynthesis article according to claim 24, wherein the reinforcing means is present in an amount up to 70% by weight relative to the total weight of the biomaterial.

39. The osteosynthesis article according to claim 24, further comprising a surfactant agent or a mixture of surfactant agents, an amphiphilic molecule or a mixture of amphiphilic molecules or any other compatibilising agent or mixture of compatibilising agents.

40. The osteosynthesis article according to claim 24, wherein added water of less than 5% by weight, relative to the total weight of biomaterial.

41. The osteosynthesis article according to claim 24, wherein the semi-aromatic polyamide matrix and the reinforcing means are selected so that the dynamic mechanical properties of the biomaterial comply with a significant level of viscoelasticity at physiological temperatures and frequencies, said level being defined by a preservative modulus and a mechanical energy loss factor in the order of those of the calcified tissue.

42. The osteosynthesis article according to claim 41, wherein the preservative modulus, represented by G′, is between 100 MPa and 10 GPa (limits included), in the shearing mode.

43. The osteosynthesis article according to claim 41, wherein the mechanical energy loss factor, represented by tan δ, is greater than 10⁻³ in the shearing mode.

44. The osteosynthesis article according to claim 28, wherein Y represents an element comprising at least one saturated, linear or branched aliphatic and/or cycloaliphatic diamine having between 4 and 20 carbon atoms.

45. The osteosynthesis article according to claim 28, wherein Ar represents an element comprising at least one substituted or unsubstituted aromatic carboxylic diacid having between 8 and 22 carbon atoms.

46. The osteosynthesis article according to claim 29, wherein Y represents an element comprising at least one saturated, linear or branched aliphatic and/or cycloaliphatic diamine having between 4 and 20 carbon atoms.

47. The osteosynthesis article according to claim 29, wherein Ar represents an element comprising at least one substituted aromatic carboxylic diacid having between 8 and 22 carbon atoms.

48. The osteosynthesis article according to claim 29, wherein X represents an element comprising at least one lactam and/or at least one carboxylic alpha-omega aminoacid, the lactam and/or the carboxylic alpha-omega aminoacid having between 6 and 18 carbon atoms.

49. The osteosynthesis article according to claim 29, wherein X represents an element U.V and the linear or branched diamine U has between 4 and 20 carbon atoms.

50. The osteosynthesis article according to claim 29, wherein X represents an element U.V and the linear or branched carboxylic diacid V has between 6 and 20 carbon atoms.

51. The osteosynthesis article according to claim 28, wherein the number of carbon atoms of the element Y is between 6 and 12 carbon atoms.

52. The osteosynthesis article according to claim 28, wherein one at least of the element Y is selected from the group consisting of: 1,6-hexamethylene diamine, 1,9-nonane diamine, 2-methyl-1,8-octane diamine, 1,10-decane diamine, 1,12-dodecane diamine and their mixtures.
